(19) United States
(12) Patent Application Publication
(10) Pub. No.: US 2022/0031562 A1
(43) Pub. Date: Feb. 3, 2022

(54) AUTOMATIC LIQUID OUTLET STRUCTURE AND BEAUTY MASSAGE INSTRUMENT

(71) Applicant: Xiamen Solex High-Tech Industries Co., Ltd., Xiamen (CN)

(72) Inventors: Qiuhua WANG, Xiamen (CN); Boren Chen, Xiamen (CN); Tingke Liu, Xiamen (CN); Mingfu Zhang, Xiamen (CN)

(21) Appl. No.: 17/504,020

(22) Filed: Oct. 18, 2021

Related U.S. Application Data
(63) Continuation of application No. PCT/CN2020/085189, filed on Apr. 16, 2020.

(30) Foreign Application Priority Data
Apr. 19, 2019 (CN) 201910317539.2

(51) Int. Cl. A61H 15/02; A61H 15/00; A61F 13/40

(52) U.S. Cl. CPC A61H 15/02; A61H 15/0092; A61M 2205/054; A61H 2015/0064; A61H 2201/105; A61M 35/006

(57) ABSTRACT

The present disclosure discloses an automatic liquid outlet structure and a beauty massage apparatus, and the automatic liquid outlet structure comprises a rolling massage mechanism, a liquid storage mechanism, and a liquid adsorption mechanism. The liquid adsorption mechanism is disposed between the rolling massage mechanism and the liquid storage mechanism. A first side of the liquid adsorption mechanism is in communication with the liquid storage mechanism, and a second side of the liquid adsorption mechanism is in contact with the rolling massage mechanism. The liquid storage mechanism is configured to hold a liquid. The liquid held in the liquid storage mechanism is configured to be adsorbed into the liquid adsorption mechanism. When the rolling massage mechanism is in a rolling process, the liquid adsorbed in the liquid adsorption mechanism is transferred to the rolling massage mechanism.

### RELATED APPLICATIONS

This application claims priority to International patent application number PCT/CN2020/085189, filed Apr. 16, 2020, which claims priority to Chinese patent application number 201910317539.2, filed on Apr. 19, 2019. International patent application number PCT/CN2020/085189 and Chinese patent application number 201910317539.2 are incorporated herein by reference.

### FIELD OF THE DISCLOSURE

The present disclosure relates to personal care equipment, and in particular to a massage apparatus.

### BACKGROUND OF THE DISCLOSURE

With the development of society and the improvement of living standards, people are not only concerned about food, clothing, housing, and transportation, but also concerned about personal care. Caring about beauty has been in existence since ancient times and is human nature. Due to the current aesthetics and the popularity of melon-shaped faces, most people like small, thin faces and sharp chins. At present, there are many face-lifting products, which use skin care products combined with micro-current to stimulate the skin, tighten the skin, and massage the skin.

At the same time, in order to increase the effectiveness of massage, beauty massage apparatuses on the market now also have a function of discharging beauty essences. That is, the beauty essences are automatically applied onto the skin during the massage, and the skin is squeezed during the massage to speed up absorption of the beauty essences. However, the fluid outlet structure of the beauty essences in the prior art generally uses one rolling ball to seal the fluid outlet of the fluid outlet structure. When the beauty massage apparatuses are tilted, the rolling ball rolls to open the fluid outlet, resulting in a fluid discharge. This structure has relatively high requirements for the inclination angle of the beauty massage apparatuses, and with a change of the inclination angle of the beauty massage apparatuses, the degree to which the fluid outlet is opened also different, resulting in the beauty essences being unevenly applied. Thereby, the beauty essences will be partially applied on the skin, or there will be too much essence on some parts of the skin.

### BRIEF SUMMARY OF THE DISCLOSURE

The present disclosure provides an automatic liquid outlet structure for use during a personal care process to realize uniformly discharging a beauty essence, of which a flow rate is independent of an inclination angle of the beauty massage apparatus.

In order to solve the technical problem, a first technical solution of the present disclosure is as follows.

An automatic liquid outlet structure comprises a rolling massage mechanism, a liquid storage mechanism, and a liquid adsorption mechanism. The liquid adsorption mechanism is disposed between the rolling massage mechanism and the liquid storage mechanism. A first side of the liquid adsorption mechanism is in communication with the liquid storage mechanism, and a second side of the liquid adsorption mechanism is in contact with the rolling massage mechanism. The liquid storage mechanism is configured to hold a liquid. The liquid held in the liquid storage mechanism is configured to be adsorbed into the liquid adsorption mechanism. When the rolling massage mechanism is in a rolling process, the liquid adsorbed in the liquid adsorption mechanism is transferred to the rolling massage mechanism.

In a preferred embodiment, when the rolling massage mechanism is in the rolling process, the rolling massage mechanism squeezes the liquid adsorption mechanism so as to enable the liquid adsorbed in the liquid adsorption mechanism to be squeezed out and to be adsorbed onto a surface of the rolling massage mechanism.

In a preferred embodiment, the rolling massage mechanism comprises one or more rolling balls and a holding frame, and the one or more rolling balls rotate during the rolling process.

In a preferred embodiment, the liquid adsorption mechanism is a sponge.

In a preferred embodiment, the liquid storage mechanism comprises a reservoir, a visible window, a liquid feeding opening, and a sealing plug for sealing the liquid feeding opening.

In a preferred embodiment, the sponge is disposed in the reservoir, and the holding frame is disposed on a top of the sponge. When the one or more rolling balls are in the rolling process, the one or more rolling balls squeeze the sponge so as to enable the liquid adsorbed in the sponge to be squeezed out and to be adsorbed onto a surface of the one or more rolling ball.

In a preferred embodiment, the one or more rolling balls are two rolling balls. The holding frame is an oval disk, and the oval disk comprises two supporting holes spaced apart from each other along a long axis of the oval disk in a symmetrical manner and two blocks extending downward and spaced apart from each other along a short axis of the oval disk. A shape of the sponge is matched with a shape of the holding frame. The sponge comprises two contact surfaces spaced apart from each other along a long axis of the sponge and two grooves spaced apart from each other along a short axis of the sponge. Motion of the two rolling balls is respectively limited in the two supporting holes, and the two rolling balls are closely attached to the two contact surfaces. The two blocks are disposed in the two grooves of the sponge.

In a preferred embodiment, the two blocks are in the shape of hollow cylinders.

In a preferred embodiment, the liquid is a beauty essence.

In a preferred embodiment, the one or more rolling balls are one or three rolling balls, and the holding frame correspondingly comprises one or three supporting holes and is a round disk or an oval disk. A shape of the sponge is matched with a shape of the holding frame.

A second technical solution of the present disclosure is as follows.

A beauty massage apparatus comprises an automatic liquid outlet structure. The automatic liquid outlet structure comprises a rolling massage mechanism, a liquid storage mechanism, and a liquid adsorption mechanism. The liquid adsorption mechanism is disposed between the rolling massage mechanism and the liquid storage mechanism. A first side of the liquid adsorption mechanism is in communication with the liquid storage mechanism, and a second side of the liquid adsorption mechanism is in contact with the rolling massage mechanism. The liquid storage mechanism is configured to hold a liquid, and the liquid held in the liquid storage mechanism is configured to be adsorbed into the liquid adsorption mechanism. When the rolling massage mechanism is in a rolling process, the liquid adsorbed in the liquid adsorption mechanism is transferred to the rolling massage mechanism.

In a preferred embodiment, the beauty massage apparatus is configured to generate a microcurrent.

In a preferred embodiment, the beauty massage apparatus comprises a handheld handle and a power supply. The power supply is disposed in the handheld handle.

In a preferred embodiment, when the rolling massage mechanism contacts skin of a user, a circuit loop is defined by a power supply, the rolling massage mechanism, and the skin of the user.

Compared with the existing techniques, the technical solution has the following advantages.

In this present disclosure, the beauty essence is adsorbed into the sponge, the rolling massage mechanism squeezes the sponge in the rolling process, and the beauty essence is squeezed out and adsorbed onto the one or more rolling balls. As a result, when the one or more rolling balls of the rolling massage mechanism are in the rolling process, the beauty essence can be applied onto a user's skin. This structure takes advantage of an adsorption effect of the sponge so as to enable the liquid storage mechanism in which the beauty essence is held to be always open, and the beauty essence flowing out from the liquid storage mechanism does not directly flow onto the user's skin, but is absorbed by the sponge. In this way, a speed at which the beauty essence flows into the sponge is roughly equal to a speed at which the beauty essence that is squeezed out flows from the sponge onto the one or more rolling balls so as to enable the user to use a massage apparatus having the automatic liquid outlet structure in any posture. Thereby, the disadvantages of the uneven application of the beauty essence and using in specific postures are avoided.

### BRIEF DESCRIPTION OF THE DRAWINGS

FIG. 1 illustrates a perspective view of a beauty massage apparatus in Embodiment 2 of the present disclosure.

FIG. 2 illustrates a top view of the beauty massage apparatus with an upper portion, shown in FIG. 4, of the beauty massage apparatus having been removed, illustrating a negative terminal being connected to a handle electrode and a positive terminal being connected to one or more rolling balls in Embodiment 2 of the present disclosure.

FIG. 3 illustrates an exploded view of an automatic liquid outlet structure in Embodiment 1 of the present disclosure.

FIG. 4 illustrates a cross-section view of the beauty massage apparatus in Embodiment 2 of the present disclosure.

### DETAILED DESCRIPTION OF THE EMBODIMENTS

The present disclosure will be further described below in combination with the accompanying drawings and embodiments.

### Embodiment 1

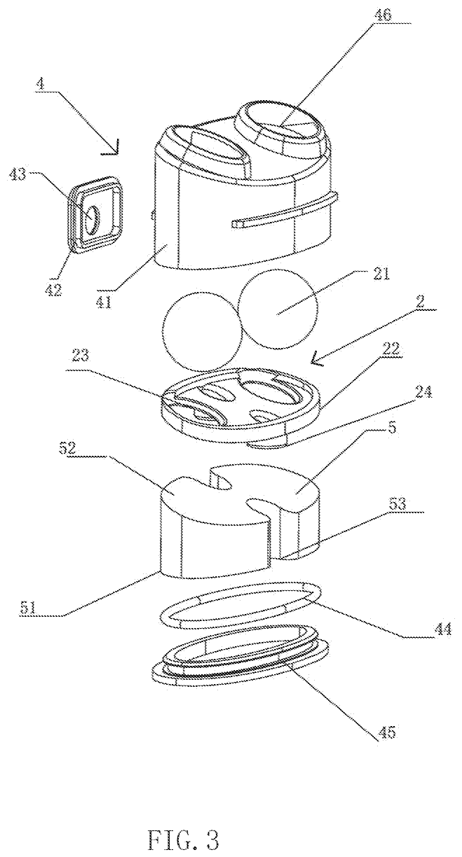

Referring to FIG. 3, an automatic liquid outlet structure is provided and comprises a rolling massage mechanism 2, a liquid storage mechanism 4, and a liquid adsorption mechanism 5.

The liquid adsorption mechanism 5 is disposed between the rolling massage mechanism 2 and the liquid storage mechanism 4. A first side of the liquid adsorption mechanism 5 is in communication with the liquid storage mechanism 4, and a second side of the liquid adsorption mechanism 5 is in contact with the rolling massage mechanism 2.

A liquid held in the liquid storage mechanism 4 is configured to be adsorbed into the liquid adsorption mechanism 5. When the rolling massage mechanism 2 is in a rolling process, the liquid adsorbed in the liquid adsorption mechanism 5 is configured to be transferred to the rolling massage mechanism 2.

In this embodiment, the liquid adsorption mechanism 5 is a sponge 51. The rolling massage mechanism 2 comprises one or more rolling balls 21 and a holding frame 22. The liquid storage mechanism 4 comprises a reservoir 41, a visible window 42, a liquid feeding opening 43, and a sealing plug 47 for sealing the liquid feeding opening 43. The sponge 51 is disposed in the reservoir 41, and the holding frame 22 is disposed on a top of the sponge 51. When the one or more rolling balls 21 are in the rolling process, the one or more rolling balls 21 squeeze the sponge 51 so as to enable the liquid adsorbed in the sponge 51 to be squeezed out and to be adsorbed onto a surface of the one or more rolling balls 21.

In this embodiment, the one or more rolling balls 21 are two rolling balls 21. The holding frame 22 is an oval disk, and the oval disk comprises two supporting holes 23 spaced apart from each other along a long axis of the oval disk in a symmetrical manner and two blocks 24 extending downward and spaced apart from each other along a short axis of the oval disk.

A shape of the sponge 51 is matched with a shape of the holding frame 22, and the sponge 51 comprises two contact surfaces 52 spaced apart from each other along a long axis of the sponge 51 and two grooves 53 spaced apart from each other along a short axis of the sponge 51.

Motion of the two rolling balls 21 is respectively limited in the two supporting holes 23, and the two rolling balls 21 are closely attached to the two contact surfaces 52. The two blocks 24 of the holding frame 22 are disposed in the two grooves 53 of the sponge 51, and the two blocks 24 can be in the shape of hollow cylinders.

In this embodiment, the liquid is a beauty essence.

The liquid storage mechanism 4 further comprises a sealing ring 44 and a bottom cover 45. A top surface of the reservoir 41 comprises two through holes 46. The two rolling balls 21, the holding frame 22, and the sponge 51 are in turn disposed in the reservoir 41, and the bottom cover 45 is sealingly connected to the reservoir 41 using the sealing ring 44.

In some embodiments, the one or more rolling balls 21 can be one or three rolling balls 21, and the holding frame 22 correspondingly comprises one or three supporting holes 23.

In the present disclosure, the beauty essence is adsorbed into the sponge 51, the rolling massage mechanism 2 squeezes the sponge 51 in the rolling process, and the beauty essence is squeezed out and adsorbed onto the one or more rolling balls 21. As a result, when the one or more rolling balls 21 of the rolling massage mechanism 2 are in the rolling process, the beauty essence can be applied onto a user's skin. This structure takes advantage of an adsorption effect of the sponge 51 so as to enable the liquid storage mechanism 4 in which the beauty essence is held to be always open, and the beauty essence flowing out from the liquid storage mechanism 4 does not directly flow onto the user's skin, but is absorbed by the sponge 51. In this way, a speed at which the beauty essence flows into the sponge 51 is roughly equal to a speed at which the beauty essence that is squeezed out flows from the sponge 51 onto the one or more rolling balls 21 so as to enable the user to use a massage apparatus having the automatic liquid outlet structure in any posture. Thereby, the disadvantages of the uneven application of the beauty essence and using in specific postures are avoided.

### Embodiment 2

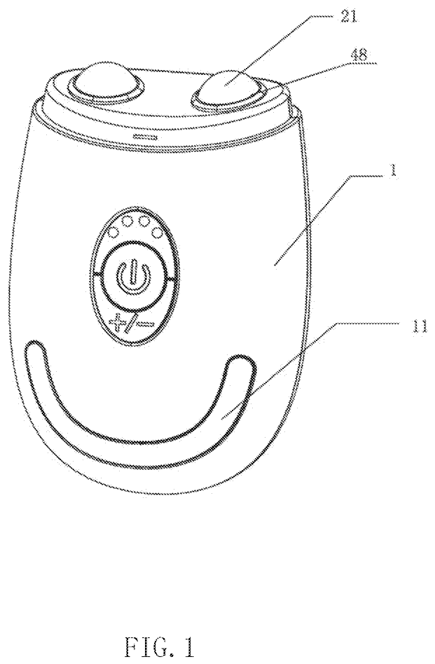

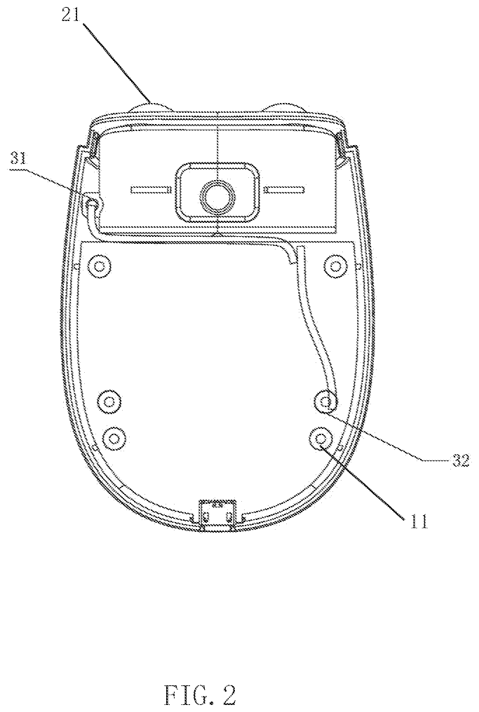

Referring to FIGS. 1-4, a beauty massage apparatus is provided and comprises a handheld handle 1 and an automatic liquid outlet structure in Embodiment 1.

In this embodiment, a power supply 3 is disposed in the handheld handle 1. The handheld handle 1 comprises a handle electrode 11, and a contact electrode is defined by a portion of the one or more rolling balls 21 protruding out of a housing of the reservoir 41. The power supply 3 is connected to each of the handle electrode 11 and the one or more rolling balls 21 using conductors. The handle electrode 11 is connected to a negative terminal 32 of the power supply 3, and the one or more rolling balls 21 are connected to a positive terminal 31 of the power supply 3. The one or more rolling balls 21 directly contact the user's skin while the user holds the handheld handle 1 so as to form a circuit loop defined by the power supply 3, the handle electrode 11, the contact electrode defined by the one or more rolling balls 21, and the user's body due to the user's body functioning as a conductor. A microcurrent generated by the circuit loop stimulates the user's body. With massage and kneading on the user's skin applied by the one or more rolling balls 21 and the stimulation by the microcurrent, the massaging care is coordinated to achieve the effect of caring for the user's body. The safety current of the human body is less than 10 mA. In order to ensure the intensity of the current, which can penetrate the user's thick epidermal cells and reach the dermis, and to ensure that the current sensed by the user is comfortable, the current range of the general household beauty instrument does not exceed 0.4 mA. In this embodiment, a maximum output current of the beauty massage apparatus is 0.28 mA, which guarantees the current sensed by the user is comfortable and ensures the safety of the current. This part belongs to the prior art and is not the main point of protection of the present disclosure, so it is only briefly explained.

In this embodiment, the power supply 3 is a lithium battery having a voltage of 3.7 V, which can output a total of 4 levels of constant output current values: 0.06 mA, 0.13 mA, 0.2 mA, and 0.28 mA. In order to achieve constant current output, a voltage of a circuit board will be adjusted through circuit feedback data. The circuit board will recognize and feedback a load current of the circuit loop in real time, and the circuit board will also adjust the voltage in real time to achieve the level of constant output current within an interval value.

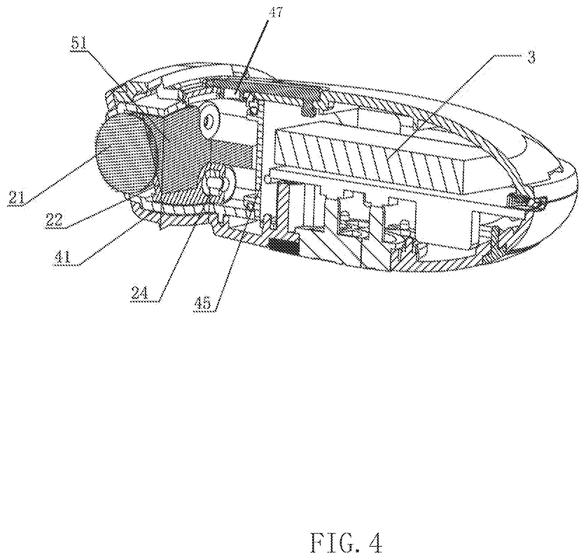

Further referring to FIGS. 3-4, in this embodiment, in order to achieve the beauty essence being applied onto the user's skin while the one or more rolling balls 21 roll during a massage process of the beauty massage apparatus, the automatic liquid outlet structure is applied.

Due to the liquid adsorption mechanism 5 directly being in communication with the liquid storage mechanism 4, the beauty essence held in the liquid storage mechanism 4 can be temporarily stored in the liquid adsorption mechanism 5. Due to the liquid adsorption mechanism 5 maintaining contact with the one or more rolling balls 21, the one or more rolling balls 21 squeezes the liquid adsorption mechanism 5 during the rolling process so as to enable the beauty essence to be squeezed out and to be adsorbed onto the one or more rolling balls 21. Furthermore, due to the one or more rolling balls 21 rotating during the rolling process, the surface of the one or more rolling balls 21 can adsorb the beauty essence, and the beauty essence can be even applied onto the user's skin when the one or more rolling balls 21 contact the user's skin.

In this embodiment, the liquid adsorption mechanism 5 is the sponge 51. The liquid storage mechanism 4 comprises the reservoir 41, the visible window 42, the liquid feeding opening 43, and the sealing plug 47 for sealing the liquid feeding opening 43. The visible window 42 is used for enabling the user to observe how much of the liquid remains in the reservoir 41 so as to enable the user to determine whether it is necessary to add more of the liquid. The liquid feeding opening 43 is an entrance for the user to add the liquid. The sealing plug 47 is used for sealing and closing the liquid feeding opening 43 when the liquid is not required to be added, so as to ensure that the liquid does not flow out of the liquid feeding opening 43.

It should be pointed out that the beauty massage apparatus is used as an example in this embodiment. In actual application, the automatic liquid outlet structure can be applied to other rolling massage apparatus. The liquid that flows out can be various other liquids in addition to the beauty essence, such as essential oils or other liquids according to the needs of the massage. All of the above are simple replacements of this embodiment and will not be repeated here.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the appended claims and the specification of the present disclosure.

What is claimed is:

1. An automatic liquid outlet structure, comprising:
a rolling massage mechanism,
a liquid storage mechanism, and
a liquid adsorption mechanism, wherein:
the liquid adsorption mechanism is disposed between the rolling massage mechanism and the liquid storage mechanism,
a first side of the liquid adsorption mechanism is in communication with the liquid storage mechanism,
a second side of the liquid adsorption mechanism is in contact with the rolling massage mechanism,
the liquid storage mechanism is configured to hold a liquid,
the liquid held in the liquid storage mechanism is configured to be adsorbed into the liquid adsorption mechanism, and
when the rolling massage mechanism is in a rolling process, the liquid adsorbed in the liquid adsorption mechanism is transferred to the rolling massage mechanism

2. The automatic liquid outlet structure according to claim 1, wherein:
when the rolling massage mechanism is in the rolling process, the rolling massage mechanism squeezes the liquid adsorption mechanism so as to enable the liquid adsorbed in the liquid adsorption mechanism to be squeezed out and to be adsorbed onto a surface of the rolling massage mechanism.

3. The automatic liquid outlet structure according to claim 1, wherein:
the rolling massage mechanism comprises one or more rolling balls and a holding frame, and
the one or more rolling balls rotate during the rolling process.

4. The automatic liquid outlet structure according to claim 3, wherein:
the liquid adsorption mechanism is a sponge.

5. The automatic liquid outlet structure according to claim 4, wherein:
the liquid storage mechanism comprises a reservoir, a visible window, a liquid feeding opening, and a sealing plug for sealing the liquid feeding opening.

6. The automatic liquid outlet structure according to claim 5, wherein:
the sponge is disposed in the reservoir,
the holding frame is disposed on a top of the sponge, and
when the one or more rolling balls are in the rolling process, the one or more rolling balls squeeze the sponge so as to enable the liquid adsorbed in the sponge to be squeezed out and to be adsorbed onto a surface of the one or more rolling ball.

7. The automatic liquid outlet structure according to claim 6, wherein:
the one or more rolling balls are two rolling balls,
the holding frame is an oval disk,
the oval disk comprises two supporting holes spaced apart from each other along a long axis of the oval disk in a symmetrical manner and two blocks extending downward and spaced apart from each other along a short axis of the oval disk,
a shape of the sponge is matched with a shape of the holding frame,
the sponge comprises two contact surfaces spaced apart from each other along a long axis of the sponge and two grooves spaced apart from each other along a short axis of the sponge,
motion of the two rolling balls is respectively limited in the two supporting holes and the two rolling balls are closely attached to the two contact surfaces, and
the two blocks are disposed in the two grooves of the sponge.

8. The automatic liquid outlet structure according to claim 7, wherein:
the two blocks are in the shape of hollow cylinders.

9. The automatic liquid outlet structure according to claim 8, wherein:
the liquid is a beauty essence.

10. The automatic liquid outlet structure according to claim 6, wherein:
the one or more rolling balls are one or three rolling balls,
the holding frame correspondingly comprises one or three supporting holes and is a round disk or an oval disk, and
a shape of the sponge is matched with a shape of the holding frame.

11. A beauty massage apparatus, comprising:
an automatic liquid outlet structure, wherein:
the automatic liquid outlet structure comprises a rolling massage mechanism, a liquid storage mechanism, and a liquid adsorption mechanism,
the liquid adsorption mechanism is disposed between the rolling massage mechanism and the liquid storage mechanism,
a first side of the liquid adsorption mechanism is in communication with the liquid storage mechanism,
a second side of the liquid adsorption mechanism is in contact with the rolling massage mechanism,
the liquid storage mechanism is configured to hold a liquid,
the liquid held in the liquid storage mechanism is configured to be adsorbed into the liquid adsorption mechanism, and
when the rolling massage mechanism is in a rolling process, the liquid adsorbed in the liquid adsorption mechanism is transferred to the rolling massage mechanism

12. The beauty massage apparatus according to claim 11, wherein:
the beauty massage apparatus is configured to generate a microcurrent.

13. The beauty massage apparatus according to claim 12, comprising:
a handheld handle, and
a power supply, wherein:
the power supply is disposed in the handheld handle.

14. The beauty massage apparatus according to claim 12, wherein:
when the rolling massage mechanism contacts skin of a user, a circuit loop is defined by a power supply, the rolling massage mechanism, and the skin of the user.
